(19) United States
(12) Patent Application Publication
(10) Pub. No.: US 2011/0262907 A1
(43) Pub. Date: Oct. 27, 2011

(54) PROCECURE FOR PREPARING A PROCESSED VIRTUAL ANALYSIS IMAGE

(75) Inventor: Eric Peltier, Clamart (FR)

(73) Assignee: NOVACYT, Velizy Villacoublay (FR)

(21) Appl. No.: 13/126,650

(22) PCT Filed: Oct. 28, 2009
(86) PCT No.: PCT/FR2009/052086

§ 371 Date: Jun. 29, 2011

(30) Foreign Application Priority Data
Oct. 31, 2008 (FR) 0857438

(51) Int. Cl. C12Q 1/68; C12Q 1/02

(52) U.S. Cl. 435/6.1; 435/29

(57) ABSTRACT

This method includes the following steps: carrying out a processing of the specimen so as to make it possible to differentiate the pathological cells from the healthy cells of the specimen; and performing an acquisition of images of the specimen disposed on an analysis plate so as to obtain a plurality of images each representing a zone of the analysis plate, the images placed side by side forming an image of the whole of the specimen so as to create a virtual analysis plate. The method furthermore includes the following step: performing on the virtual analysis plate a processing of the images acquired so as to obtain a virtual restitution of the colors and of the intensity of the colors of the cytoplasm and/or of the nucleus, the colors and the intensity being able to be modified according to the preferences of the person in charge of the analysis.

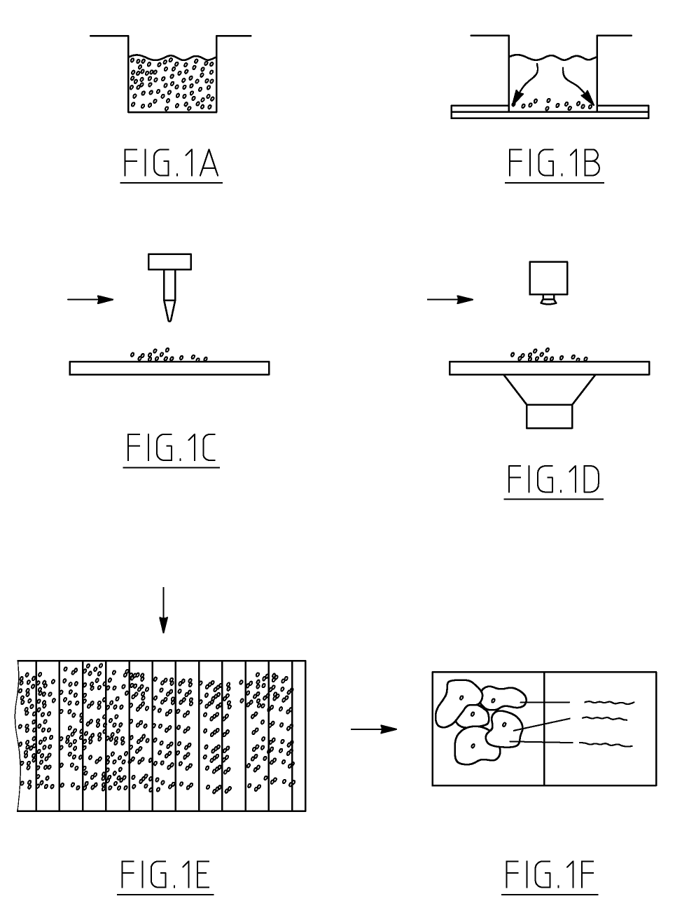

The present invention relates to a method for preparing a virtual analysis plate, of a cytological or histological specimen disposed on an analysis plate so as to allow cell analysis of said specimen, of the type comprising the following steps:
performing a processing of the specimen, said processing being performed in order to allow differentiation of pathological cells from healthy cells of the specimen,
carrying out an acquisition of images of the specimen disposed on the analysis plate so as to obtain a plurality of images each representing a zone of the analysis plate, said images placed side by side forming an image of the whole of the specimen so as to create a virtual analysis plate.

The invention particularly applies to cytological or histological analysis methods.

The invention also relates to a method for preparing a virtual analysis plate of a specimen in order to allow its cell analysis, notably according to an analysis method as described above.

The analysis of specimens is for example used for diagnosing pathologies, for example from cells sampled by smearing (cervical, vaginal or other smears), by puncture of organs (breast, thyroid, ganglion or other organs) or further by collection (urine, broncho-alveolar lavage or other liquids) for detecting any type of pathology and more particularly pre-cancerous or cancerous conditions.

The specimens are examined by specialized and trained observers for detecting cells which may be pathological in a specimen disposed on an analysis plate or slide. In order to allow detection of potentially pathological cells, the specimen undergoes a treatment such as staining allowing identification i.a. of the characteristics of the nucleus and of the cytoplasm of the cells in order to contribute to localization and to the diagnosis of pathological cells. When the specimen is observed, the potentially pathological cells then show differences in dye affinities, in both size and shape, both at the nucleus and at the cytoplasm, with respect to normal cells.

The analysis may be accomplished manually, without any particular assistance. In this case, the physician or the specialized technician has the specimen plates pass under a microscope and observes each of them in order to detect morphological abnormalities indicating pathological cells which may correspond to a pre-cancerous or cancerous condition for example. Such an analysis method is tedious and consumes a great deal of time. Further, it does not provide satisfactory results with especially a number of <<false negatives>> estimated to be about 30%, i.e. specimens considered as normal whereas there exist a pathology in the patient, notably a pre-cancerous or cancerous pathology with risks of subsequent development of cancer in a wrongly reassured patient.

In order to improve the results of the analyses, it was proposed to improve the sampling, i.e. the number of cells, their fixing, their staining and their spreading over the analysis plate, but also to assist the physician or the specialized technician in their analysis for example by means of computer analysis means, such as image processing software packages and other means.

For this purpose, a still or video camera is used for acquiring images of the different zones of the specimen placed on the analysis plate and for transferring the data of these images to a computer system which then operates on a <<virtual>> analysis plate.

This computer system allows processing of the signal, pre-processing of the images and comparative analysis of the images with optionally newly created or existing databases in order to accelerate the analysis process and to thereby allow analysis of a larger number of specimens and in order to provide assistance to the physician or specialized technician. The images of a specimen are for example examined automatically and if certain zones having an abnormality are found, the corresponding images are forwarded to a physician or specialized technician who may then determine whether these zones show pathological cells or not. The physician or specialized technician therefore only observes abnormal zones without analyzing the zones considered as normal by the computer system. With such a method it is actually possible to accelerate the analysis and make the diagnosis more reliable.

However the physician or specialized technician then has no longer the opportunity of observing normal specimens or those exhibiting minor morphological modifications, which is detrimental to his/her appreciation of specimens and especially for his/her learning curve or even for preserving his/her diagnostic acuity. Indeed, the analysis of specimens is based on the training and practice of the physician or specialized technician in examining specimens and in comparing normal zones and zones having abnormalities. Suppressing these practices by computer-aided analysis may lead to physicians or specialized technicians losing their skills and thereby causing errors in analysis.

Further the digitization operation may take a considerable time because of the treatments applied on the slide before digitization.

The invention aims at finding a remedy to the aforementioned drawbacks by proposing an analysis method which, by being assisted in order to allow a notable gain in time, notably during the digitization operation, also gives the possibility to physicians or specialized technicians of practicing the observation of both normal and abnormal specimens.

For this purpose, the invention relates to a method of the aforementioned type further comprising the following steps:
carrying out on the virtual analysis plate, processing of the acquired images in order to obtain a virtual restoration of the colors and of the intensity of the colors of the cytoplasm and/or of the nucleus, said colors and said intensity may be modified according to the preferences of the person in charge of the analysis.

Such a method gives the possibility of producing in a particularly simple way an image of a specimen with which possibly pathological cells may be detected and a great amount of information may be acquired on the specimen, as this will be described later on.

According to other characteristics of the preparation method:
the method further comprises a step for superimposition of the data of the images acquired during the acquisition of the images and of those of the acquired images modified during the processing of the acquired images,
the treatment of the specimen is a nuclear or cytological coloration/staining step, said treatment being laid out so as to make the cytoplasm almost transparent and to enhance the contrast between the nucleus and/or cytoplasm RNA of the cells and the cytoplasm,
the processing of the acquired images corresponds to virtual re-coloration of the Papanicolaou, Schorr, May Grunwald Giemsa or Giemsa type of these acquired images,
the virtual re-coloration level of the acquired images is adjustable,
the method comprises a step for displaying the virtual analysis plate,
the display of the analysis plate is accompanied by the display of information on the displayed specimen area and/or on the whole of the specimen and/or on the patient on which the specimen was taken and/or on the result of complementary examinations conducted on the specimen,
the method comprises a step for enlarging the virtual analysis plate in order to allow viewing of a detail of said plate,
the enlargement is adjustable.

Other aspects and advantages of the invention will become apparent upon reading the following description, given as an example and made with reference to the appended drawing which is a schematic illustration of the different steps of the method for preparing a virtual analysis plate according to the invention.

With reference to the FIGURE, a method for preparing a virtual analysis plate is described with view to cell analysis aided by a computer system. By virtual plate, is meant a set of pieces of information and of numerical data grouped together, relating to a specimen.

The specimen is for example obtained by smearing (cervical, vaginal or other smears), by puncture of organs (breast, thyroid, ganglion or other organs) or further by collection (urine, broncho-alveolar lavage or other fluids). During a first step A, the specimen is suspended in a solution, for example, in a tube or a sampling flask.

During a step B, the specimen is disposed on an analysis plate. In a known way, deposition of the cells onto the plate is for example accomplished by decantation. The specimen is poured into a decantation chamber, the bottom of which is opened onto the analysis plate. Absorption means allow gradual absorption of the solution as the cells are deposited on the analysis plate. Such a deposition method is known and will not be described in detail here.

During a step C, the specimen undergoes a first treatment aiming at staining/coloring the nucleis, the DNA and/or RNA of the cells by very significantly enhancing the contrast relative to the cytoplasm of these same cells. Such a staining allows proper segmentation of nuclei in order to conduct a morphological study and for accomplishing quantification of the DNA (in the scope of the ploidy for example) and/or of the RNA in order to locate potentially pathological cells.

This staining is for example carried out automatically by means of an automaton provided with pipeting means used for both putting the specimen into solution and for depositing the cell suspension on an analysis plate.

The step for decanting the cell suspension may be performed before, after or between the staining and/or coloration steps described above.

Other colorations used in the state of the art may be used, but they have drawbacks. Thus, stoichiometric coloration may be contemplated, which provides coloration of the cells proportionally to the amount of DNA, which allows its quantification, and therefore location and analysis of the pathological cells within the scope of the ploidy. However, this particular coloration, when this is Feulgen's coloration for example, is <<physically>> incompatible with Papanicolaou's coloration and therefore requires that a new spreading of the cells be accomplished on the slide for the analysis of the physician or specialized technician. Certain industrialists have attempted to associate stoichiometric coloration with Papanicolaou's coloration and have therefore used a coloration containing a thionine and requiring fixing with methanol which is toxic, and especially which modifies Papanicolaou's coloration in its interpretation, notably with nuclei for which chromatin appears too <<black>> for fine analysis of the composition of said nuclei, and therefore causes a difficulty in analysis for the diagnosis of pre-cancerous or cancerous conditions.

During step D, the analysis plate comprising the specimen, colored with a known nuclear dye or with a known cytological coloration, but modified for making the cytoplasm almost transparent, is subject to illumination in white light in order to acquire the images of the specimen by means of the image acquisition device. By acquiring images in white light, it is possible to obtain images of the specimen colored by nuclear coloration or modified cytological coloration keeping cytoplasms almost transparent in order to increase the contrast with the nuclei.

With the image acquisition device, it is possible to <<scan>> the specimen with very high resolution and obtain from a plate, images in white light. The analysis plate is scanned line by line or fields by fields as proposed by many industrialists. Thus, each acquired image represents a strip of small width of the analysis plate or fields of view. The images placed side by side give the possibility of obtaining an image of the whole of the sample plate and therefore of the whole of the specimen in order to form a virtual analysis plate, as illustrated in step E of the FIGURE. Thus, the image, obtained with a single and same device and in a very simple way, is a true representation of the specimen associating a large number of pieces of information in white light.

The digital data obtained with the device in white light undergo computer processing in order to obtain a modified virtual analysis plate formed with the image having undergone virtual re-coloration of the Papanicolaou, Schorr, May Grunwald Giemsa or Giemsa type, aiming at coloring the cells as conventionally described and known for cytological analysis by one skilled in the art. According to a computer processing mode, the virtual re-coloration of Papanicolaou, obtained as known for a long time, and the semiology of which, widely described in the literature, allows possible recognition of cytoplams and nuclear abnormalities for example corresponding to the presence of pre-cancerous or cancerous cells. With this re-coloration it is also possible to recognize different types of cells and their number in order to determine the representative quality of the specimen and, for example, to define whether the specimen is representative or not. With this virtual re-coloration it is possible to analyze the virtual plate with a cell analysis method as described below.

Cell analysis is accomplished by having the images of the specimen examined by the physician or the specialized technician in charge of detecting pathological cells, in order to suggest a diagnosis which will optionally trigger more extensive examinations or even a treatment. For reasons of safety, the presence of the physician or of the specialized technician is mandatory so that the detection of possibly pathological cells cannot be entirely automated.

During a step F, the images of the modified virtual analysis plate formed with the image having undergone virtual re-coloration are projected on a display means such as a screen. Virtual restoration of the colors and of their intensity both for the cytoplasm and for the nucleus, may be adjusted so as to correspond as close as possible to the habits of each reader, i.e. the physician or the specialized technician, in terms of coloration intensity and quality.

And then these images pass under the eyes of the physician or the specialized technician for examination. The scrolling of the images is organized by the computer system and is accomplished automatically. Each image is displayed for a predetermined time calculated in order to allow the physician or the specialized technician to observe the whole of each projected image and to detect a possible abnormality in an image. The time for displaying each image may be adjusted by the physician or the specialized technician depending on his/her skills or depending on other information. The information on the patient may be associated with a virtual analysis plate by entering this information into a database and associating it with the image of the specimen corresponding to the patient from whom this specimen was taken.

The display of the images may be accompanied by the display of information on the displayed sampling area and/or on the whole of the specimen and/or on the patient from whom the specimen was taken and/or on the result of complementary examinations conducted on the specimen, notably molecular biology examinations.

The images which pass by, are therefore those of the modified virtual analysis plate, formed with the image having undergone virtual re-coloration of the Papanicolaou, Schorr, May Gruwald Giemsa or Giemsa type. The images under this coloration enable detection of the possible presence of abnormalities for example corresponding to the presence of pre-cancer or cancerous cells and the semiology of which, known for long time, is widely described in the literature. Further, as indicated above, the Papanicolaou coloration allows verification of whether the specimen actually meets the Bethesda criteria, within the scope of a uterine cervical smear for example, and is therefore a valid specimen or not. The Bethesda criteria may be verified automatically by the computer system by means of an image analysis software package. This software package may for example carry out counting of the healthy or pathological cells. The counting of all the cells of the image allows verification of whether at least 5,000 cells have actually been sampled and if certain types of cells, a testimony of the good quality of the specimen, such as endocervical cells or from the junction, have been taken. The software package may also acquire other information on the cell spreading. This information is linked to the image by the computer system in order to complete the virtual analysis plate.

The image displayed under virtual re-coloration, and for which scrolling has been interrupted, is associated with the image of the same zone without the virtual re-coloration. With this display, the physician or the specialized technician may refine his/her analysis of the display cells and either confirm or not whether some are possibly pathological. Further, with the displaying of the image without any virtual re-coloration, other information may be displayed simultaneously, such as quantitative data, spectra or information on the patient from whom the specimen was taken, etc. This finer analysis coupled with automated stopping of the scrolling allows the number of false negatives to be reduced. Further, within the scope of the uterine cervical smear for example, with the control of the diagnosis by the physician or the specialized technician of the zones selected by the system, it is possible to preserve the specificity level of the cytological diagnosis which is close to 95% in this case. Consequently, the sensitivity and specificity criteria of the screening smear become closer and higher.

During this step, the physician or the specialized technician may freely carry out magnification of particular zones of the displayed image, both on the image with virtual re-coloration and on the image without virtual re-coloration. The physician or the specialized technician may then switch from the image with virtual re-coloration to the image without virtual re-coloration as they see fit.

The method described above allows rapid and effective analysis of specimens, reducing the risk of <<false negatives>>.

Further, the experience of physicians or the specialized technicians in terms of diagnostic quality and especially specificity is preserved because of the recognition of the colors and of their intensity related to the virtual images of processed and displayed cytological and/or histological preparations.

With the method it is also possible to adapt the analysis plate to the preferences of the physician or the specialized technician in charge of the analysis. Indeed, the physician or the specialized technician may select the intensity and the coloration of the virtual analysis plate at will.

1. A method for preparing a virtual analysis plate of a cytological or histological specimen disposed on an analysis plate with view to allowing cell analysis of said specimen, comprising the following steps:
carrying out a processing for staining the specimen, said processing being carried out so as to make it possible to differentiate the pathological cells from the healthy cells of the specimen,
performing an acquisition of images of the specimen disposed on the analysis plate, so as to obtain a plurality of images each representing a zone of the analysis plate, said images placed side by side forming an image of the whole of the specimen so as to create a virtual analysis plate,
characterized in that it further comprises the following step:
performing on the virtual analysis plate a treatment for re-coloration of the acquired images so as to obtain virtual restoration of the colors and of the intensity of the colors of the cytoplasm and/or of the nucleus, said colors and said intensity being able to be modified according to the preferences of the person in charge of the analysis.

2. The preparation method according to claim 1, characterized in that it further comprises a step for superimposing the data of the images acquired during acquisition of the images and of those of the acquired images modified during the processing of the acquired images.

3. The preparation method according to claim 1, characterized in that the treatment of the specimen is a nuclear or cytological coloration step, said treatment being laid out so as to make the cytoplasm almost transparent and to enhance the contrast between the nucleus and/or the cytoplasm RNA of the cells and the cytoplasm.

4. The preparation method according to claim 1, characterized in that the processing of the acquired images corresponds to virtual re-coloration of the Papanicolaou, Schorr, May Grunwald Giemsa or Giemsa type of these acquired images.

5. The preparation method according to claim 1, characterized in that the virtual re-coloration level of the acquired images is adjustable.

6. The preparation method according to claim 1, characterized in that it comprises a step for displaying the virtual analysis plate.

7. The preparation method according to claim 6, characterized in that the display of the analysis plate is accompanied by the display of information on the displayed specimen area and/or on the whole of the specimen and/or on the patient from whom the specimen was taken and/or on the result of complementary examinations conducted on the specimen.

8. The preparation method according to claim 1, characterized in that it comprises a step for enlarging the virtual analysis plate in order to allow viewing of a detail of said plate.

9. The preparation method according to claim 8, characterized in that the enlargement is adjustable.

10. The preparation method according to claim 2, characterized in that the treatment of the specimen is a nuclear or cytological coloration step, said treatment being laid out so as to make the cytoplasm almost transparent and to enhance the contrast between the nucleus and/or the cytoplasm RNA of the cells and the cytoplasm.

11. The preparation method according to claim 2, characterized in that the processing of the acquired images corresponds to virtual re-coloration of the Papanicolaou, Schorr, May Grunwald Giemsa or Giemsa type of these acquired images.

12. The preparation method according to claim 2, characterized in that the virtual re-coloration level of the acquired images is adjustable.

13. The preparation method according to claim 2, characterized in that it comprises a step for displaying the virtual analysis plate.

14. The preparation method according to claim 2, characterized in that it comprises a step for enlarging the virtual analysis plate in order to allow viewing of a detail of said plate.
